(19) United States
(12) Patent Application Publication
(10) Pub. No.: US 2026/0083753 A1
(43) Pub. Date: Mar. 26, 2026

(54) Compositions for the Treatment of Metabolic Syndrome and Other Metabolic Disorders, and Methods of Manufacture

(71) Applicant: Florida Research Group, LLC, Tampa, FL (US)

(72) Inventor: Kotha SEKHARAM, Tampa, FL (US)

(21) Appl. No.: 19/274,778

(22) Filed: Jul. 21, 2025

Related U.S. Application Data
(60) Provisional application No. 63/697,787, filed on Sep. 23, 2024.

(51) Int. Cl. A61K 31/56; A61K 47/10; A61K 47/18; A61K 47/22; A61K 47/32; A61P 3/00

(52) U.S. Cl. CPC A61K 31/56; A61K 47/10; A61K 47/18; A61K 47/22; A61K 47/32; A61P 3/00

(57) ABSTRACT

The subject invention provides improved compositions for metabolic syndrome and methods of preparing the compositions via, for example, hot-melt extrusion. The resulting extrudate has improved solubility, bioavailability and therapeutic effect for treatment of metabolic syndrome at a low dose.

### CROSS REFERENCE TO A RELATED APPLICATION

This application claims the benefit of U.S. Provisional Application Ser. No. 63/697,787, filed Sep. 23, 2024, which is incorporated herein by reference in its entirety.

### BACKGROUND OF THE INVENTION

Metabolic syndrome is a cluster of five conditions that together raise the risk of coronary heart disease, diabetes, stroke, and other serious health problems.

A diagnosis of metabolic syndrome is made based on the presence of three or more of the following abnormalities/risk factors: high blood glucose, low levels of HDL (“good”) cholesterol in the blood, high levels of triglycerides in the blood, large waist circumference or “apple-shaped” body and high blood pressure. Although each of these is a risk factor for cardiovascular disease, when a person has three or more of these abnormalities, the chance of developing a serious cardiovascular condition increases.

Patients with metabolic syndrome are estimated to have a 2-fold increased risk of atherosclerotic cardiovascular diseases and a 5-fold increased risk of diabetes mellitus, as compared to the general population. Metabolic syndrome is also associated with accelerated atherosclerosis, premature atherosclerotic cardiovascular diseases, and early onset type II diabetes mellitus.

The risks associated with metabolic syndrome can be reduced significantly with lifestyle changes, including losing weight, increasing physical activity, eating a heart-healthy diet that is rich in whole grains, fruits, vegetables and fish; and by managing blood glucose, blood cholesterol and blood pressure.

If a change to healthy lifestyle cannot be achieved or is insufficient, there is a need for specific medications for each condition. For example, ACE inhibitors, angiotensin receptor blockers, diuretics, and beta blocker to lower blood pressure; statins and niacin to manage cholesterol levels; GLP-1 agonists such as liraglutide, semaglutide, and tirzepatide, to lower blood sugar levels.

Odd chain fatty acids are used to treat metabolic syndrome [U.S. Pat. No. 10,449,171 B2].

U.S. Pat. No. 9,636,314 B2 teaches a method of using steviol, isosteviol or stevioside for the treatment of non-insulin dependent diabetes mellitus, hypertension and/or metabolic syndrome.

Various health supplements are being used to manage specific conditions related to metabolic syndrome. For example, chokeberry, citrus bergamia, green tea, mulberry, omega 3 fatty acids, vitamin B3, and zinc to manage cholesterol; curcumin and ginseng to reduce fasting glucose; garlic, phosphorus, pomegranate, quercetin, and vitamin D, to reduce blood pressure; and resveratrol, to reduce body weight.

Several other botanicals have been reported to be useful for treating metabolic syndrome, including extract of evodamine fructus, citrus unshiu Markovich and imperatae rhizome as active ingredients [KR patent 102009011]; soybean hypocotyl oil as an active ingredient [WO 2018/01226]; methoxyl stilbene compound [CN 101269054]; astaxanthin containing extract [WO 2007/037438]; methoxyl flavonoids [CN 101269061]; vinegar processed ginseng [KR 1020050102017]; ethyl cellulose [WO 2008/051793]; corosolic acid [WO 2007/094497]; β-1,3-1,4-glucan [WO 2010/116866].

Botanicals such as berberine, curcumin, gingerol, gymnemic acid, gymnemagenin and mangiferin have also used in the treatment of metabolic syndrome.

3β-hydroxyurs-12-en-28-oic acid, also called ursolic acid, Prunol, Malol, or Urson is a naturally-derived lipophilic pentacyclic triterpene that is found mostly in rosemary, marjoram, lavender, thyme, organum, and apple fruit peel and has a chemical formula of C₃₀H₄₈O₃, and molecular weight of 456.7 g/mol. Uroslic acid has a wide range of potential therapeutic benefits as an anti-oxidant, anti-inflammatory, anti-cancer, anti-allergic, hepatoprotective, gastroprotective, hypolipidemic, hypoglycemic, lipolytic anti-obesity, anti-atherogenic and immunomodulatory effects.

The effect of ursolic acid on nuclear receptors was reviewed by Kadasah et. al; (2023) who report potential health benefits including treatment of conditions via nuclear receptor pathways.

Ursolic acid has an anti-inflammatory effect on male Wistar rats with HCHFD-induced MS by decreasing serum TNF-α and IL-6, increasing adiponectin and upregulating adipose tissue PPAR-γ mRNA levels, respectively [Oluwatosin Omodara et. al 2023].

Alejandra M Ramirez-Rodriguez et. al; (2017) reported that 150 mg of ursolic acid administered orally once a day for 12 weeks lead to transient remission of metabolic syndrome, by reducing body weight, BMI, waist circumference and fasting glucose, as well as increasing insulin sensitivity. A difference in lipid profile, including triglycerides, total cholesterol, and HDL-c circulating concentrations was not observed.

Jose G. C. Cione et. al (2021) investigated the effects of ursolic acid supplementation associated with a combined exercise program on metabolic syndrome components in postmenopausal women in a randomized controlled trial. No significant effects between groups were observed for waist circumference and other secondary outcomes. Furthermore, remission of metabolic syndrome occurred in 38.5% of the participants in both groups.

Ursolic acid supplementation prevented the development of metabolic syndrome in male Wistar rats fed with HCHFD for 20 weeks. [Omodara et. al; 2022]. The Ursolic acid supplementation did not, however, promote additional metabolic syndrome profile improvement after 8 weeks of free-living intervention. No significant effects between groups were observed for waist circumference and other secondary outcomes. Furthermore, remission of metabolic syndrome (according to the NCEP ATP III criteria) occurred in 38.5% of the participants in both groups. [No additional effects of ursolic acid supplementation associated with combined exercise program on metabolic syndrome of postmenopausal women: A double-blind, randomized, placebo-controlled trial, José G. C. Cione et al: Clinical Nutrition ESPEN Volume 44, August 2021, 143-149].

3β-Hydroxyolean-12-en-28-oic acid, also called as oleanolic acid (OA) is another triterpenoid isomeric compound that exists widely in food, medicinal herbs and other plants, which has been extensively studied to treat metabolic syndrome and cardiovascular diseases. In spite of positive results in the laboratory, OA, like ursolic acid, also has limitations in the treatment of metabolic syndrome and cardiovascular diseases. OA has low bioavailability and is insoluble in water [Luo, Q.; Wei, Y et. al; 2024].

3β-hydroxyurs-12-en-28-oic acid and 3β-Hydroxyolean-12-en-28-oic acid are both water-insoluble. Limited solubility, poor bioavailability and rapid metabolism limited their clinical applications for therapeutic use.

Because of this low bioavailability, published literature with these pentacyclic triterpenoids reports inconsistent dosages ranging from 150 mg to 4,500 mg per dose.

Variation in Ursolic acid dose, as reported
in studies with human and animal studies

| Medical | | |
|---|---|---|
| condition | Dose | Reference |
| Metabolic | Orally 150 mg/dose for 12 weeks | Ram.rez-Rodriguez |
| syndrome | (human) | et al., 2017 |
| Obesity | 0.5% UA for 6 weeks and 20 and 40 | Chu et al. 2015 |
| | μM for 4 hours (rats) | |
| Obesity | 0.14 or 0.27% for 6 weeks (rats) | Kunkel et al. 2012 |
| Obesity | 0.125, 0.25, and 0.5% for 6 weeks | Li et al. 2014 |
| | (in rats) | |
| Obesity | 1 and 2% for 9 weeks (in mice) | Kazmi et al. 2013 |
| Obesity | 250 mg/kg for 8 weeks (rats) | Zhang et al. 2016 |
| Diabetes | 200 mg/kg per day for 6 weeks | Yu et al. 2015 |
| Cognitive | 100 mg/kg for 3 weeks (rats) | Wu et. al; 2013 |
| Aging | 200 mg/kg (twice a day for 7 days) | Bakhtiari et al. |
| | (mice) | 2015 |
| Performance | 3 g after exercise. Resistance- | Church et al. 2016 |
| | trained healthy men | |
| Performance | 450 mg/day for 8 weeks | Bang et al. 2017 |

There is a need for dependable, consistent, therapeutically effective dose.

Depending on the nature of the material, dosage could be reduced by altering certain parameters. Several techniques have been considered, including chemical modification (prodrug, salt formation), physical modification (nano crystals, co-crystals, loading on porous materials, solid dispersions), and carrier system (cyclodextrins, emulsions, microemulsions, liposomes).

A reduction in particle size improves the dissolution rate significantly. Wet milling and nano-technology are two techniques that can be applied to improve absorption to a certain extent; however, difficulties in scalability and contaminants from milling equipment limit commercial applications.

Solubilization can be improved by particle size reduction, pH adjustment, salt formation, solid dispersion, complexation, co-solvency, micellization, or a combination effect of any of the above. Processing methods involving usage of solvents is highly unfavorable due to residual solvents, environmental concerns and challenges in scalability limit their application in production process.

Trikatu is a blend of 3 spices, dried fruits of Piper nigrum (Maricha), Piper longum (Peepli), and dried rhizomes of Zingiber officinale (Sunthi) has been in use as an adjunct to herbal medication in Ayurveda.

Piperine has been reported as a natural bioenhancer. Its bioenhancing effect has been shown by either action on a metabolizing enzyme, enhancing drug transport, affecting blood supply to the gastrointestinal tract (GIT), and/or membrane fluidity.

Long pepper, commonly called “Pippal” in Sanskrit, is an important medicinal spice with similar alkaloidal constituent and similar functionality of Piperine.

Ursolic acid (UA) was combined with piperine to enhance the hepatoprotective potential of UA and to study the pharmacodynamic and pharmacokinetic interaction [Sayan Biswas, Amit Kar, Nanaocha Sharma, Pallab K. Haldar & Pulok K. Mukherjee (2021) Synergistic effect of ursolic acid and piperine in CCl4 induced hepatotoxicity, Annals of Medicine, 53:1, 2009-2].

Ginger (Z. officinale) has a powerful effect on gastrointestinal tract mucous membrane. It regulates the intestinal function to facilitate absorption.

The solubility of a compound in amorphous form (which is an unstable form) is higher than the more stable crystalline form because the Gibbs free energy is higher. In practice, the challenge is not only converting to amorphous form, but also in maintaining in that ‘unstable’ state until its end use.

Thus, there is a need for developing formulations with enhanced solubility and ultimately the bioavailability of therapeutic compounds for therapeutic uses.

### BRIEF SUMMARY OF THE INVENTION

The present invention provides methods of improving the utility of insoluble compound 3β-hydroxyurs-12-en-28-oic acid by combining the compound with at least one of other insoluble compound such as piperine, capsaicin, and/or gingerol and processing the composition through hot melt extrusion using mechanical and thermal energies. The resulting extrudate has improved solubility, bioavailability and therapeutic effect for treatment of metabolic syndrome, and other metabolic disorders, including at low dose.

In one embodiment, the subject invention provides a method for preparing a composition having improved solubility and bioavailability, the method comprising:
(1) blending
   (a) at least one of the following:
      3β-hydroxyurs-12-en-28-oic acid (UA), an isomer, or a salt thereof; and
      3β-Hydroxyolean-12-en-28-oic acid (OA), an isomer or a salt thereof;
   (b) at least one modulator extract selected from the group consisting of piperine, capsaicin, and gingerol; and
   (c) a water-soluble polymer having a glass transition temperature lower than 170° C.;
(2) melt extruding the blend of (1) at a temperature of about 60° C. to about 180° C. to obtain an extrusion product; and
(3) optionally, grinding the extrusion product and mixing the ground extrusion product with a pharmaceutical acceptable carrier or excipient to obtain the composition.

In specific embodiments, such composition is free from any solvent associated with the processing steps.

In one embodiment, the subject invention provides a composition comprising:
(a) at least one of the following:
   UA, an isomer, or a salt thereof; and
   OA, an isomer or a salt thereof;
(b) at least one modulator extract selected from the group consisting of piperine, capsaicin, and gingerol;
(c) a water-soluble polymer having a glass transition temperature lower than 170° C.; and
(d) optionally, a pharmaceutically acceptable carrier.

In a specific embodiment, the composition comprises UA, OA, an isomer, or a salt thereof in amorphous form.

In specific embodiments, the water-soluble polymers that can be used include polyvinyl caprolactam, polyvinyl acetate-polyethylene glycol graft copolymer, polyvinylpyrrolidone-vinyl acetate copolymer, polyethylene glycol, amino methacrylate copolymer, and hypromellose acetate succinate.

In one embodiment, such composition is therapeutically effective to manage metabolic syndrome at a very low dose in a subject such as mammals, including humans.

In one embodiment, the subject invention provides a method for treating a metabolic disorder, the method comprising administering to a subject having been diagnosed with the metabolic disorder a therapeutically effective amount of a composition of the subject invention.

In one embodiment, the subject invention provides a method for treating metabolic syndrome, the method comprising administering to a subject having been diagnosed with metabolic syndrome a therapeutically effective amount of a composition of the subject invention.

### BRIEF DESCRIPTION OF THE DRAWINGS

FIG. 1: X-ray diffractometry of [A] untreated Ursolic acid indicating crystalline nature [B] treated Ursolic acid indicating amorphous nature [C] treated Ursolic acid −2 indicating amorphous nature.

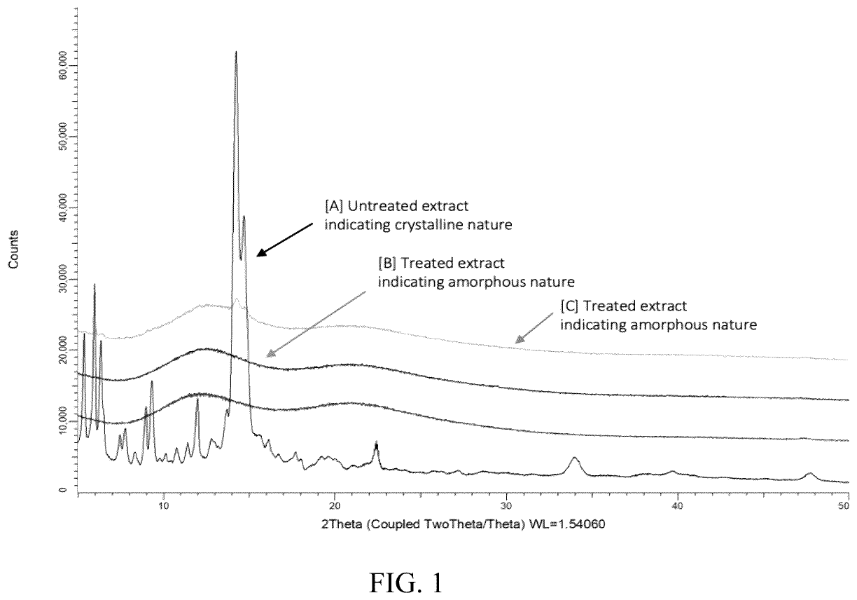

### DETAILED DESCRIPTION OF THE INVENTION

The subject invention provides improved compositions for treating metabolic disorders, including, metabolic syndrome, and methods of preparing the compositions via, for example, hot-melt extrusion. In one embodiment, the subject invention also provides methods for treating metabolic syndrome, or other metabolic disorder. In one embodiment, the subject invention further provides a diet supplement comprising the improved composition of the subject invention.

In one embodiment, the subject invention provides a method for preparing an improved composition for treating a metabolic disorder, wherein the method comprises blending one or more active pharmaceutical ingredients (APIs) with at least one modulator extract and a polymer; and melt extruding the blend by melting and extruding.

In one embodiment, the improved composition comprises the one or more APIs, at least one modulator extract and the polymer. In one embodiment, the product of extrusion comprises the improved composition, in particular, the one or more APIs in a solid dispersion.

As used herein, a solid dispersion is a system in which one or more active ingredients are molecularly distributed into a carrier matrix. The formulation of an API into a solid dispersion converts the poorly water-soluble crystalline form to the amorphous form, which increases its solubility. Advantageously, the solid dispersions significantly increase the dissolution rate of the API compared to that of the pure drug or physical mixtures. Thus, the API having a poor solubility or being insoluble has improved solubility once prepared by using the process of the subject invention.

In specific embodiments, the one or more APIs are selected from 3β-hydroxyurs-12-en-28-oic acid (UA), 3β-hydroxyolean-12-en-28-oic acid (OA), isomers and salts thereof. In specific embodiments, UA and/or OA is a component of a botanical extract.

In one embodiment, the improved composition comprises one or more APIs in a weight percentage amount in a range of, for example, about 0.1% to 60%, about 0.1% to 50%, about 0.1% to 40%, about 0.1% to 30%, about 0.1% to 20%, about 0.1% to 10%, about 0.2% to 55%, about 0.2% to 50%, about 0.2% to 40%, about 0.2% to 30%, about 0.2% to 20%, about 0.2% to 10%, about 0.5% to 50%, about 0.5% to 40%, about 0.5% to 30%, about 0.5% to 20%, about 0.5% to 10%, about 0.5% to 5%, about 1% to 50%, about 1% to 45%, about 1% to 40%, about 1% to 35%, about 1% to 30%, about 1% to 25%, about 5% to 50%, about 5% to 40%, about 5% to 25%, about 10% to 50%, about 10% to 40%, about 10% to 30%, about 10% to 20%, about 15% to 50%, about 20% to 50%, about 25% to 50%, about 30% to 50%, or about 40% to 50%.

In certain embodiments, the API may be found in the form of one or more pharmaceutically acceptable salts, esters, derivatives, analogs, prodrugs, and solvates thereof. The APIs contemplated within the scope of the invention may be in their free acid, free base, or a pharmaceutically-acceptable salt form. They may also be derivatives or prodrugs of a given pharmaceutical.

The APIs may be used in a variety of application modalities, including, for example, oral delivery as tablets, capsules or suspensions; pulmonary and nasal delivery; topical delivery as emulsions, ointments or creams; transdermal delivery; and parenteral delivery as suspensions, microemulsions or depot. As used herein, the term “parenteral” includes, for example, subcutaneous, intravenous, intramuscular, or infusion routes of administration.

### Preparation Process

In one embodiment, the subject invention provides a method for preparing a composition with improved solubility and bioavailability, the method comprising:
(1) blending
   (a) at least one of the following:
      3β-hydroxyurs-12-en-28-oic acid (UA), an isomer, or a salt thereof; and
      3β-Hydroxyolean-12-en-28-oic acid (OA), an isomer or a salt thereof;
   (b) at least one modulator extract; and
   (c) a polymer;
(2) melt extruding the blend of (1) to obtain an extrusion product; and
(3) optionally, grinding the extrusion product and mixing the ground extrusion product with a pharmaceutical acceptable carrier or excipient to obtain the composition.

In a specific embodiment, the extrusion product may be used directly as a composition for treating metabolic disorders or metabolic syndrome.

In specific embodiments, the modulator extract is selected from the group consisting of piperine, capsaicin, and gingerol.

In certain embodiments, the polymer is thermoplastic, and has a suitable glass transition temperature and a high thermal stability. The polymer also has no toxic properties and has high biocompatibility. In preferred embodiments, the polymer is a water-soluble polymer. In specific embodiments, the polymer is a water-soluble polymer having a glass transition temperature ≤170° C., 160° C., 150° C., or 140° C.

In specific embodiments, the method comprises using a water-soluble polymer is selected from the group consisting of polyvinyl caprolactam, polyvinyl acetate-polyethylene glycol graft copolymer, polyvinylpyrrolidone-vinyl acetate copolymer, polyethylene glycol, Eudragit® EPO, and hypromellose acetate succinate.

In specific embodiments, the melt extruding step is carried out using a hot melt extruder. In certain embodiments, the hot-melt extruder is not limited insofar as it is an extruder having a structure capable of applying a shear force to the blend described herein with a piston or screw while heating for melting and kneading, and then extruding through a die or an element alike.

In certain embodiments, the extruder may be any commercially-available model equipped to handle feed and having, for example, one or more of the following: a solid conveying zone, one or multiple heating zones, and an extrusion die. In a preferred embodiment, a twin-screw extruder is used according to the subject invention to obtain a more uniform extrusion product.

In specific embodiments, the melt extruding step is carried out at a hot melt temperature. In certain embodiments, the hot-melt temperature is in a temperature range that allows the blend for hot-melt extrusion to be melted and extruded smoothly, while avoiding excessive thermal decomposition of the API (e.g., UA and/or OA), modulator extract and the polymer.

In specific embodiments, the hot-melt temperature is from about 50 to 250° C., from about 60 to 240° C., from about 60 to 230° C., from about 60 to 220° C., from about 60 to 210° C., from about 60 to 200° C., from about 60 to 190° C., from about 60 to 180° C., from about 60 to 170° C., from about 60 to 160° C., from about 60 to 150° C., from about 60 to 140° C., from about 60 to 130° C., from about 60 to 120° C., from about 60 to 110° C., from about 70 to 240° C., from about 80 to 230° C., from about 90 to 220° C., from about 100 to 210° C., from about 110 to 200° C., from about 120 to 190° C., from about 130 to 180° C., from about 140 to 170° C., from about 100 to 180° C., or from about 90 to 190° C.

In certain embodiments, the extruder comprises at least four heating zones, each of which can have the same or different temperature. In specific embodiments, the temperature of the heating zones is sequentially lowered. It is particularly advantageous for the extruder to possess multiple separate temperature-controllable heating zones.

In certain embodiments, the extrusion process of the subject invention comprises passing a blend through the at least four heating zones while it is melted, wherein the temperature of each heating zone is sequentially lowered.

In certain embodiments, after the extrusion, the extrusion product is cooled after the die outlet port by natural cooling at room temperature or by blowing cold air to minimize the thermal decomposition of the API (e.g., UA and/or OA) and to prevent recrystallization when the API (e.g., UA and/or OA) is in an amorphous form.

In certain embodiments, the extrusion product after cooling may be optionally pelletized into pellets by using a cutter, or optionally ground to regulate the particle size until it becomes granular or powdery. In specific embodiments, for grinding, an impact grinder such as a jet mill, a knife mill or a pin mill may be used.

In certain embodiments, the extrusion product may be shaped, molded, chopped, ground, molded, spheronized into beads, cut into strands, tableted or otherwise processed to the desired physical form. In certain embodiments, the extrusion product may have a desired shape such as circular, square, columnar, and film shape. In a specific embodiment, the extrusion product may be formulated to a powdery composition.

In one embodiment, the subject invention provides a method for preparing an improved composition for treating metabolic disorders, wherein the method comprises:
blending (a) one or more of 3β-hydroxyurs-12-en-28-oic acid (UA), 3β-hydroxyolean-12-en-28-oic acid (OA), isomers and salts thereof with (b) at least one modulator extract, and (c) a polymer having a glass transition temperature lower than 170° C.;
melt extruding the blend to obtain an extrusion product; and
optionally, grinding the extrusion product and mixing with a pharmaceutically acceptable carrier or excipient.

In certain embodiments, the method for preparing a composition of the subject invention comprises passing the blend through at least four heating zone of a extruder, wherein each heating zone has a temperature independently ranging from, for example, about 50 to 250° C., from about 60 to 240° C., from about 60 to 230° C., from about 60 to 220° C., from about 60 to 210° C., from about 60 to 200° C., from about 60 to 190° C., from about 60 to 180° C., from about 60 to 170° C., from about 60 to 160° C., from about 60 to 150° C., from about 60 to 140° C., from about 60 to 130° C., from about 60 to 120° C., from about 60 to 110° C., from about 70 to 240° C., from about 80 to 230° C., from about 90 to 220° C., from about 100 to 210° C., from about 110 to 200° C., from about 120 to 190° C., from about 130 to 180° C., from about 140 to 170° C., from about 100 to 180° C., or from about 90 to 190° C.

In one embodiment, the subject invention provides a method for preparing an improved composition for treating metabolic disorders, wherein the method comprises:
(i) blending (a) one or more of UA, OA, isomers and salts thereof with (b) at least one modulator extract selected from the group consisting of piperine, capsaicin, and gingerol, and (c) a water-soluble polymer having a glass transition temperature lower than 170° C.;
(ii) melt extruding the blend of (i) at a temperature of about 60° C. to about 180° C.; and
(iii) grinding and mixing with excipients to form a capsule, tablet, chewable tablet, soft chew, gummy, lozenge, bar, oral paste, suppository or powder composition.

In specific embodiments, the composition of the subject invention is free from any solvent associated with processing steps.

In specific embodiments, the composition of the subject invention comprises 3β-hydroxyurs-12-en-28-oic acid and/or 3β-hydroxyolean-12-en-28-oic acid in an amorphous form.

In preferred embodiments, the blend of 3β-hydroxyurs-12-en-28-oic acid and/or 3β-hydroxyolean-12-en-28-oic acid, modulator and water-soluble polymer is melted while it passes through at least four heating zones, wherein a temperature of the heating zones is sequentially lowered.

In preferred embodiments, the 3β-hydroxyurs-12-en-28-oic acid is a compound of formula:

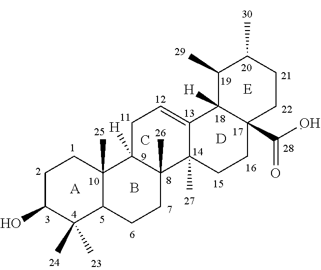

In preferred embodiments the 3-hydroxyolean-12-en-28-oic acid is a compound of formula:

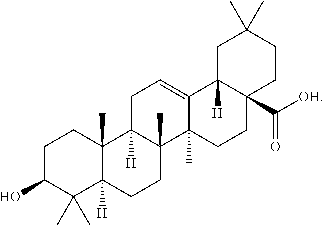

In a specific embodiment the modulator extract is piperine of formula:

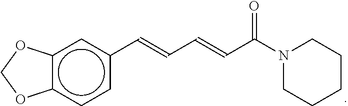

### Compositions

In one embodiment, the subject invention provides a composition comprising:
(a) at least one of the following:
   UA, an isomer, or a salt thereof; and
   OA, an isomer or a salt thereof;
(b) at least one modulator extract;
(c) a polymer; and
(d) optionally, a pharmaceutically acceptable carrier.

The composition of the subject invention is therapeutically effective to manage a metabolic disorder in a subject, e.g., a mammal. Advantageously, in preferred embodiments, the composition of the subject invention can be used at low dosages to treat metabolic syndrome.

In specific embodiments, the modulator extract is selected from the group consisting of piperine, capsaicin, and gingerol.

In certain embodiments, the polymer is thermoplastic, and have a suitable glass transition temperature and a high thermal stability. The polymer also has no toxic properties and has a high biocompatibility. In preferred embodiments, the polymer is a water-soluble polymer. In specific embodiments, the polymer is a water-soluble polymer having a glass transition temperature ≤170° C., 160° C., 150° C., or 140° C.

In specific embodiments, the composition comprises a water-soluble polymer is selected from the group consisting of polyvinyl caprolactam, polyvinyl acetate-polyethylene glycol graft copolymer, polyvinylpyrrolidone-vinyl acetate copolymer, polyethylene glycol, Eudragit® EPO, and hypromellose acetate succinate.

In one embodiment, the composition of the subject invention comprises UA, OA, an isomer or a salt thereof in a weight percentage amount in a range of, for example, about 0.1% to 60%, about 0.1% to 50%, about 0.1% to 40%, about 0.1% to 30%, about 0.1% to 20%, about 0.1% to 10%, about 0.2% to 55%, about 0.2% to 50%, about 0.2% to 40%, about 0.2% to 30%, about 0.2% to 20%, about 0.2% to 10%, about 0.5% to 50%, about 0.5% to 40%, about 0.5% to 30%, about 0.5% to 20%, about 0.5% to 10%, about 0.5% to 5%, about 1% to 50%, about 1% to 45%, about 1% to 40%, about 1% to 35%, about 1% to 30%, about 1% to 25%, about 5% to 50%, about 5% to 40%, about 5% to 25%, about 10% to 50%, about 10% to 40%, about 10% to 30%, about 10% to 20%, about 15% to 50%, about 20% to 50%, about 25% to 50%, about 30% to 50%, or about 40% to 50%.

In one embodiment, the improved composition comprises at least one modulator extract in a weight percentage amount in a range of, for example, about 0.01% to 30%, about 0.01% to 25%, about 0.01% to 20%, about 0.01% to 15%, about 0.01% to 10%, about 0.01% to 5%, about 0.01% to 1%, about 0.1% to 25%, about 0.1% to 20%, about 0.1% to 15%, about 0.1% to 10%, about 0.1% to 5%, about 0.2% to 25%, about 0.2% to 20%, about 0.2% to 15%, about 0.2% to 10%, about 0.2% to 5%, about 0.5% to 25%, about 0.5% to 20%, about 0.5% to 15%, about 0.5% to 10%, about 0.5% to 5%, about 1% to 20%, about 1% to 10%, about 1% to 5%, about 5% to 25%, or about 5% to 20%.

In one embodiment, the improved composition comprises a polymer in a weight percentage amount in a range of, for example, about 1% to 95%, about 1% to 90%, about 1% to 85%, about 1% to 80%, about 1% to 75%, about 1% to 70%, about 1% to 65%, about 1% to 60%, about 1% to 55%, about 1% to 50%, about 1% to 45%, about 1% to 40%, about 1% to 35%, about 1% to 30%, about 1% to 25%, about 1% to 20%, about 2% to 95%, about 2% to 90%, about 2% to 85%, about 2% to 80%, about 2% to 75%, about 2% to 70%, about 2% to 65%, about 2% to 60%, about 2% to 55%, about 2% to 50%, about 2% to 45%, about 2% to 40%, about 2% to 35%, about 2% to 30%, about 2% to 25%, about 2% to 20%, about 5% to 90%, about 5% to 85%, about 5% to 80%, about 5% to 75%, about 5% to 70%, about 5% to 65%, about 5% to 60%, about 5% to 55%, about 5% to 50%, about 5% to 45%, about 5% to 40%, about 5% to 35%, about 5% to 30%, about 5% to 25%, about 5% to 20%, about 10% to 90%, about 10% to 85%, about 10% to 80%, about 10% to 75%, about 10% to 70%, about 10% to 65%, about 10% to 60%, about 10% to 55%, about 10% to 50%, about 10% to 45%, about 10% to 40%, about 10% to 35%, about 10% to 30%, about 20% to 90%, about 20% to 85%, about 20% to 80%, about 20% to 75%, about 20% to 70%, about 20% to 65%, about 20% to 60%, about 20% to 55%, about 20% to 50%, about 20% to 45%, about 20% to 40%, about 20% to 35%, about 20% to 30%, about 30% to 90%, about 30% to 80%, about 30% to 70%, about 30% to 60%, about 30% to 50%, about 30% to 40%, about 40% to 90%, about 50% to 80%, or about 60% to 70%.

In certain embodiments, a weight ratio of the part (a) to the part (b) may be, for example, from 100:1 to 1:10, preferably, from 50:1 to 1:2, more preferably, from 10:1 to 1:1. In certain embodiments, a weight ratio of the part (a) to the part (c) may be, for example, from 100:1 to 1:100, preferably, from 50:1 to 1:50, more preferably, from 20:1 to 1:20, most preferably, from 10:1 to 1:10.

In some embodiments, the composition of the subject inventio further comprises an additive, such as a plasticizer and a surfactant for improving the extrusion property during the hot-melt extrusion. In some embodiments, the additive may also include, for example, surface active materials, anti-oxidants, stabilizing agents, solubility-enhancing agents, pH control agents and flow regulators.

As used herein, the term “plasticizer” includes all compounds capable of plasticizing the applied polymer. The plasticizer should be able to lower the glass transition temperature or softening point of the polymer in order to allow for lower processing temperature, extruder torque and pressure during the hot-melt extrusion process. Plasticizers generally broaden the average molecular weight of the polymer thereby lowering its glass transition temperature or softening point. Plasticizers also generally reduce the viscosity of a polymer melt thereby allowing for lower processing temperature and extruder torque during hot-melt extrusion.

Examples of the plasticizer include, for example, higher alcohols such as acetone, methanol, ethanol, isopropanol, cetyl alcohol, polyvinyl alcohol and stearyl alcohol; polyols such as mannitol, sorbitol and glycerin; beeswax; triethyl citrate; alkylene glycols such as polyethylene glycol and propylene glycol; triacetin; dibutyl sebacate; glycerin monostearate; and monoglycerin acetate.

Examples of the surfactant include, for example, anionic surfactants such as sodium lauryl sulfate; nonionic surfactants such as diglyceride, poloxamer, polyoxyethylene sorbitan fatty acid esters (Tween 20, 60 and 80), glycerin fatty acid esters and propylene glycol fatty acid esters; and natural surfactants such as lecithin and sodium taurocholate.

In certain embodiments, the extrusion product can be used as an oral solid preparation such as tablets, granules, fine granules and capsules, or oral film formulations, optionally combined with one or more various additives typically and conventionally used in this field such as an excipient, a binder, a disintegrant, a lubricant and an anti-aggregation agent.

Examples of the excipient include, for example, sugars such as sucrose, lactose, mannitol and glucose; starches; and crystalline celluloses.

Examples of the binder include, for example, polyvinyl alcohol, polyacrylic acid, polyvinylpyrrolidone, hydroxyethyl cellulose, hydroxypropylmethyl cellulose, hydroxypropyl cellulose, macrogols, gum arabic, gelatin and starches.

Examples of the disintegrant include, for example, low-substituted hydroxypropyl cellulose, carmellose or salt thereof, croscarmellose sodium, carboxymethyl starch sodium, crospovidone, crystalline cellulose and crystalline cellulose.carmellose sodium.

Examples of the lubricant and the anti-aggregation agent include, for example, talc, magnesium stearate, calcium stearate, colloidal silica, stearic acid, waxes, hydrogenated oil, polyethylene glycols and sodium benzoate.

In certain embodiments, the oral solid preparation obtained may be film-coated with a water-soluble coating agent such as methyl cellulose or hypromellose; or coated with an enteric coating agent such as hypromellose acetate succinate, hypromellose phthalate, or a methacrylate acrylate copolymer.

In one embodiment, the composition according to the subject invention also comprises a pharmaceutically acceptable carrier. “Pharmaceutically acceptable carrier” refers to a diluent, adjuvant or excipient with which the one or more active agents disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith.

Examples of carriers suitable for use in the pharmaceutical compositions are known in the art and such embodiments are within the purview of the invention. The pharmaceutically acceptable carriers and excipients, including, but not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

In certain embodiments, the composition is in a form of, for example, capsule, tablet, chewable tablet, soft chew, gummy, lozenge, bar, oral paste, suppository and powder.

In one embodiment, the subject invention provides a pharmaceutical composition comprising:
(a) at least one of the following:
   UA, an isomer, or a salt thereof; and
   OA, an isomer or a salt thereof;
(b) at least one modulator extract selected from the group consisting of piperine, capsaicin, and gingerol;
(c) a water-soluble polymer having a glass transition temperature lower than 170° C.; and
(d) optionally, a pharmaceutically acceptable carrier.

In further embodiments, 3β-hydroxyurs-12-en-28-oic acid, or 3β-hydroxyolean-12-en-28-oic acid is a chemical isolate, or salt, isomer or a botanical extract, botanical extract containing one or both compounds.

In preferred embodiments, the 3β-hydroxyurs-12-en-28-oic acid is a compound of formula:

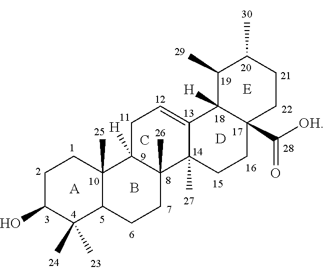

In preferred embodiments the 3-hydroxyolean-12-en-28-oic acid is a compound of formula:

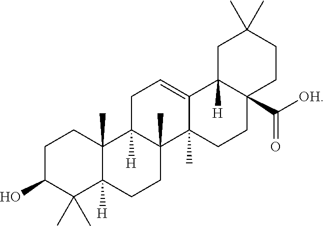

In a specific embodiment, the modulator extract is piperine of formula:

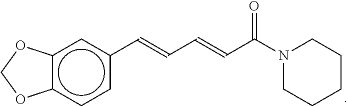

In preferred embodiments, the water-soluble polymer is selected from the group consisting of polyvinyl caprolactam, polyvinyl acetate-polyethylene glycol graft copolymer, polyvinylpyrrolidone-vinyl acetate (PVP-VA) copolymer, polyethylene glycol (PEG), Eudragit® EPO, hypromellose acetate succinate and combinations thereof.

In a specific embodiment, the water-soluble polymer is poly vinylpyrrolidonevinyl acetate copolymer.

In preferred embodiments, 3β-hydroxyurs-12-en-28-oic acid and/or 3β-hydroxyolean-12-en-28-oic acid is about 1% to 50% by weight of the composition produced according to the subject invention.

In a specific embodiment, the modulator extract is about 0.1 to about 25% by weight of the composition produced according to the subject invention.

In a specific embodiment, the water-soluble polymer is about 5% to about 90% of the weight of the composition produced according to the subject invention.

In one embodiment, the composition may be formulated for oral administration. Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder (such as microcrystalline cellulose, gelatin, a sugar, polyethylene glycol, natural and synthetic gums, polyvinylpyrrolidone, pregelatinised starch, hydroxypropyl cellulose and hydroxypropyl methylcellulose); an excipient (such as such as lactose, mannitol, xylitol, dextrose, sucrose, sorbitol, microcrystalline cellulose, starch, and dibasic calcium phosphate dehydrate); a disintegrating agent (such as alginic acid, Primogel, sodium starch glycolate, sodium carboxymethyl cellulose, calcium carboxymethyl cellulose, polyvinylpyrrolidone, methyl cellulose, microcrystalline cellulose, lower alkyl-substituted hydroxypropyl cellulose, starch, pregelatinised starch and sodium alginate); a lubricant (such as magnesium stearate, calcium stearate, zinc stearate, sodium stearyl fumarate, and mixtures of magnesium stearate with sodium lauryl sulfate); a glidant such as colloidal silicon dioxide; a sweetening agent such as sucrose or saccharin; or a flavoring agent such as peppermint, methyl salicylate, or orange flavoring. In certain embodiments, the composition is applied topically or systemically or via a combination of both. The composition may be formulated in the forms of lotion, cream, gel and the like.

In certain embodiments, the composition may be provided in the form of a dry powder, for example a powder mix of the compound in a suitable powder base such as lactose, starch, starch derivatives such as hydroxypropylmethyl cellulose and polyvinylpyrrolidone (PVP). The powder composition may be presented in unit dose form for example in capsules or cartridges of, e.g., gelatin, or blister packs from which the powder may be administered by means of an inhaler.

In one embodiment, the composition is provided in a unit dosage form, wherein the composition in desired form is divided into unit doses containing appropriate quantities of the active component. The unit dosage form can be a packaged preparation, the package containing discrete quantities such as packaged tablets, capsules, and powders in vials or ampoules. Moreover, the unit dosage form can be a capsule, tablet, cachet, or lozenge itself, or it can be the appropriate number of any of these in packaged form. In a preferred embodiment, tablet or capsule forms are for oral administration and liquid form are for intravenous administration and continuous infusion.

In one embodiment, dosage units contain about 0.01 mg to 1000 mg, about 0.01 mg to 900 mg, about 0.01 mg to 800 mg, about 0.01 mg to 700 mg, about 0.01 mg to 600 mg, about 0.01 mg to 500 mg, about 0.05 mg to 500 mg, about 0.1 mg to 400 mg, about 0.1 mg to 300 mg, about 0.1 mg to 200 mg, about 0.1 mg to 100 mg, about 0.1 mg to 90 mg, about 0.1 mg to 80 mg, about 0.1 mg to 70 mg, about 0.1 mg to 60 mg, about 0.1 mg to 50 mg, about 0.1 mg to 40 mg, about 0.1 mg to 30 mg, about 0.1 mg to 20 mg, about 0.1 mg to 10 mg, about 0.5 mg to 50 mg, about 1 mg to 40 mg, about 1 mg to 20 mg, about 1 mg to 10 mg, or about 1 mg to 5 mg of the active ingredients.

The composition of the subjection invention may also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the subject's condition and requirements.

In one embodiment, the pharmaceutically acceptable salt as used herein includes, for example, salts that have the desired pharmacological activity of the parent compound (salts which retain the biological effectiveness and/or properties of the parent compound and which are not biologically and/or otherwise undesirable). Salts, as described herein, may be prepared by conventional processes known to a person skilled in the art, for example, and without limitation, by combining the free form with an organic acid or cation exchange from other salts. Those skilled in the art will appreciate that preparation of salts may occur in situ during isolation and purification of the compounds or preparation of salts may occur by separately reacting an isolated and purified compound.

In certain embodiments, the salts are derived from sodium, potassium, ammonium, calcium, magnesium, iron, silver, zinc, or copper; particularly suitable salts include ammonium, potassium, sodium, calcium and magnesium salts.

The effective amount of said pharmaceutical composition can be administered through, for example, oral, rectal, bronchial, nasal, topical, buccal, sub-lingual, transdermal, vaginal, intramuscular, intraperitoneal, intravenous, intra-arterial, intracerebral, interaocular administration or in a form suitable for administration by inhalation or insufflation, including powders and liquid aerosol administration, or by sustained release systems such as semipermeable matrices of solid hydrophobic polymers containing the compound(s) of the invention. Administration may be also by way of other carriers or vehicles such as patches, micelles, liposomes, vesicles, implants (e.g., microimplants), synthetic polymers, microspheres, nanoparticles, and the like.

### Methods of Treatment

In one embodiment, the subject invention provides a method for treating a metabolic disorder, the method comprising administering to a subject in need of such treatment a composition of the subject invention.

Metabolic disorders are an increasingly prevalent category of disorders including Obesity [including obesity comorbidities including but not limited to metabolic syndrome, dyslipidemia, Type III dyslipidemia, hypertension, insulin resistance, diabetes (including Type 1 and Type 2 diabetes), coronary artery disease, and heart failure]; Overweightness or increased weight; Increased body mass index; Metabolic syndrome; Diabetes [including diabetes-related disorders including Type 1 diabetes (insulin-dependent diabetes mellitus, IDDM) and Type 2 diabetes (non-insulin-dependent diabetes mellitus, NIDDM)]; Diabetic complications (including, but not limited to atherosclerosis, coronary heart disease, stroke, peripheral vascular disease, nephropathy, hypertension, neuropathy and nephropathy); Impaired glucose tolerance; Elevated blood glucose; Insulin resistance; Insulin insensitivity; Hyperglycemia; Fatty liver disease; Non-alcoholic fatty liver disease; Hepatic insulin resistance; Glycosuria; Increased blood triglycerides; Increased appetite; and Dyslipidemia.

In specific embodiments, the subject has been diagnosed with a metabolic disorder.

As used herein, the term “subject” refers to an animal, needing or desiring delivery of the benefits provided by a therapeutic agent. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease, or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, “treatment” or “treating” (and grammatical variants of these terms), refers to an approach for obtaining beneficial or desired results including but not limited to therapeutic benefit. A therapeutic benefit is achieved with the eradication or amelioration of one or more of the physiological symptoms associated with the underlying condition such that an improvement is observed in the subject, notwithstanding that the subject may still be afflicted with the underlying condition. Also, the term “treatment” refers to eradicating, reducing, ameliorating, or reversing a sign or symptom of a health condition, disease, or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

In one embodiment, the subject invention provides a method for treating metabolic syndrome, the method comprising administering to a subject in need of such treatment a composition of the subject invention.

Metabolic syndrome is a combination of medical disorders that increase the risk of cardiovascular disease. A diagnosis of metabolic syndrome requires at least three of the following criteria (U.S. National Cholesterol Education Program (NCEP), 2001 and revised NCEP, 2004):
1) central obesity (waist circumference: men >102 cm (˜40 inches); women >88 cm (˜35 inches));
2) elevated triglycerides (≥150 mg/dl);
3) diminished high-density lipoprotein (HDL) cholesterol (men <40 mg/dl; women <50 mg/dl);
4) systemic hypertension (raised blood pressure) (systolic ≥130/diastolic ≥85 mm Hg); and
5) elevated fasting glucose (≥110 mg/dl) [rNCEP elevated fasting glucose (≥100 mg/dl)].

Additional criteria may support a diagnosis for metabolic syndrome or metabolic disorders, such as Body Mass Index (e.g., BMI >30 kg/m2). Metabolic syndrome may also be related to elevated total cholesterol. Elevated LDL cholesterol is marked by levels above about 100, about 130, about 160 or about 200 mg/dL. Metabolic syndrome may also be related to elevated total cholesterol. Impaired glucose intolerance is defined as a two-hour glucose level (glycemia) of about 140 to about 199 mg/dL (7.8 to 11.0 mmol) on the 75-g oral glucose tolerance test (according to WHO and ADA). Glycemia of about 200 mg/dl or greater is considered diabetes mellitus. Hyperglycemia, or high blood sugar, can be defined as a blood glucose level higher than about 7, about 10, about 15, or about 20 mmol/L. Hypoglycemia, or low blood sugar, can be defined as preprandial blood glucose below about 4 or about 6 mmol/L (72 to 108 mg/dl) or 2-hour postprandial blood glucose below about 5 or about 8 mmol/L (90 to 144 mg/dl). Insulin resistance is defined as a state in which a normal amount of insulin produces a subnormal biologic response. Insulin resistance can be measured by the hyperinsulinemic euglycemic clamp technique, Homeostatic Model Assessment (HOMA), or Quantitative insulin sensitivity check index (QUICKI). Hyperuricemia is an abnormally high level of uric acid in the blood, e.g., above 360 μmol/L (6 mg/dL) for women and 400 μmol/L (6.8 mg/dL) for men. Polycystic ovarian syndrome (PCOS) is associated with oligoovulation, anovulation, excess androgen, and/or polycystic ovaries. Metabolic syndrome may also be associated with acanthosis nigricans. Metabolic syndrome may also be associated with a pro-inflammatory state (e.g., elevated C-reactive protein levels in the blood, e.g., above 10 mg/L) and microalbuminuria (urinary albumin excretion ratio ≥20 mg/min or albumin:creatinine ratio ≥30 mg/g).

In a specific embodiment, the subject invention has been diagnosed with metabolic syndrome.

In one embodiment, the subject invention provides a method for reducing the risk of a subject for heart disease, diabetes, and stroke, the method comprising administering to the subject in need a composition of the subject invention.

In certain embodiments, the method of treatment described therein may further comprises a step of measuring/detecting the criteria and/or parameters described herein before and/or after the treatment.

In certain embodiment, the composition is therapeutically effective at about 0.01 mg to about 100 mg/kg body weight, about 0.02 mg to about 100 mg/kg body weight, about 0.05 mg to about 100 mg/kg body weight, about 0.1 mg to about 100 mg/kg body weight, about 0.5 mg to about 100 mg/kg body weight, about 1 mg to about 100 mg/kg body weight, about 10 mg to about 100 mg/kg body weight, about 20 mg to about 100 mg/kg body weight, about 30 mg to about 100 mg/kg body weight, about 40 mg to about 100 mg/kg body weight, about 50 mg to about 100 mg/kg body weight, about 0.01 mg to about 90 mg/kg body weight, about 0.05 mg to about 80 mg/kg body weight, about 0.05 mg to about 70 mg/kg body weight, about 0.05 mg to about 60 mg/kg body weight, about 0.05 mg to about 50 mg/kg body weight, about 0.05 mg to about 40 mg/kg body weight, about 0.05 mg to about 30 mg/kg body weight, about 0.05 mg to about 20 mg/kg body weight, about 0.05 mg to about 10 mg/kg body weight, about 0.05 mg to about 5 mg/kg body weight, about 0.5 mg to about 90 mg/kg body weight, about 0.5 mg to about 80 mg/kg body weight, about 0.5 mg to about 70 mg/kg body weight, about 0.5 mg to about 60 mg/kg body weight, about 0.5 mg to about 50 mg/kg body weight, about 0.5 mg to about 40 mg/kg body weight, about 0.5 mg to about 30 mg/kg body weight, about 0.5 mg to about 20 mg/kg body weight, about 1 mg to about 80 mg/kg body weight, about 1 mg to about 70 mg/kg body weight, about 1 mg to about 60 mg/kg body weight, about 1 mg to about 50 mg/kg body weight, about 1 mg to about 40 mg/kg body weight, about 1 mg to about 30 mg/kg body weight, about 1 mg to about 20 mg/kg body weight, or about 1 mg to about 10 mg/kg body weight.

In a specific embodiment, the composition is therapeutically effective at about 0.05 mg to about 50 mg/kg body weight.

In a specific embodiment, composition is therapeutically effective at about 0.5 mg to about 20 mg/kg body weight.

In a specific embodiment, composition is therapeutically effective at about 1 mg to about 10 mg/kg body weight.

In preferred embodiments, the composition is prepared as a finished product and is meant for mammals, including humans.

In preferred embodiments, the composition is ingested as an adjunct to food, and feed.

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.”

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%). Where the term “about” is used to describe target temperatures or durations of time used in certain processes, the target temperatures of durations of time can be varied within a range 0-10% around the target value (X±10%).

In the present disclosure, ranges are stated in shorthand, to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, and 0.7-1.0.

When ranges are used herein, such as for dose ranges, combinations and sub-combinations of ranges (e.g., subranges within the disclosed range), specific embodiments therein are intended to be explicitly included.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### Results

TABLE 1
Pharmacokinetic Parameters of untreated Ursolic
Acid and treated Ursolic Acid Following Oral administration
in male Beagle Dogs at 50 mg/dog

| | Untreated Ursolic acid | | Treated Ursolic acid | |
|---|---|---|---|---|
| PK Parameters | Mean | SD | Mean | SD |
| Tₘₐₓ (h)^{@} | 3 | NR | 2 | NR |
| Cₘₐₓ (ng/mL) | 12.51 | 14.03 | 58.85 | 27.41 |
| AUC_{0-24 h} (ng · h/mL) | 101.12 | 131.36 | 358.04 | 157.05 |
| AUC_{0-inf} (ng · h/mL) | 2242.83^{#} | NA | 390.96 | 158.83 |
| T_{1/2} (h) | 134.65^{#} | NA | 6.62 | 1.97 |
| MRT last | 7.46 | 4.19 | 7.61 | 1.32 |

^{@}Median;
NA: Not applicable;
^{#}one animal data;
$: two animal data

TABLE 2
Mean Plasma Concentrations of Untreated Ursolic
acid and Treated Ursolic Acid Following Oral administration
of in male Beagle Dogs at 50 mg/dog

| | | |
|---|---|---|
| Time Points | Untreated Ursolic acid | Treated Ursolic acid |

| (h) | Mean | SD | Mean | SD |
|---|---|---|---|---|
| 0 | 0 | 0 | 0 | 0 |
| 0.08 | 1.57 | 1.38 | 1.54 | 1.75 |
| 0.25 | 2.07 | 2.6 | 5.11 | 5.21 |
| 0.5 | 11.27 | 14.74 | 25.43 | 23.81 |
| 1 | 8.99 | 11.25 | 38.06 | 45.31 |
| 2 | 4.29 | 3.16 | 29.28 | 25.64 |
| 3 | 4.09 | 1.59 | 31.31 | 12.26 |
| 4 | 6.38 | 5.34 | 28.7 | 10.87 |
| 6 | 5.15 | 4.06 | 20.95 | 6.44 |
| 8 | 1.61 | 0.39 | 10.64 | 0.42 |
| 10 | 1.87 | 2.04 | 10.4 | 7.83 |
| 12 | 5.61 | 7.65 | 15.97 | 17.97 |
| 24 | 3.42 | 5.92 | 3.21 | 1.26 |

### REFERENCES

Alejandra M Ramirez-Rodriguez et. al; Randomized Controlled Trial J Med Food 2017 September; 20(9):882-886.
Bakhtiari N, Hosseinkhani S, Tashakor A, Hemmati R. Ursolic acid ameliorates agingmetabolic phenotype through promoting of skeletal muscle rejuvenation. Med Hypotheses. 2015; 85:1-6.
Bang H S, Seo D Y, Chung Y M, Kim D H, Lee S J, Lee S R, Kwak H B, Kim T N, Kim M, Oh K M, Son Y J, Kim S, Han J. Ursolic acid supplementation decreases markers of skeletal muscle damage during resistance training in resistance-trained men: a pilot study. Korean J Physiol. Pharmacol. 2017; 21:651-656.
Church D D, Schwarz N A, Spillane M B, McKinley-Barnard S K, Andre T L, Ramirez A J, Willoughby D S. 1-Leucine increases skeletal muscle IGF-1 but does not differentially increase Akt/mTORC1 signaling and serum IGF-1 compared to ursolic acid in response to resistance exercise in resistance-trained men. J Am Coll Nutr. 2016; 35:627-638.
Chu X, He X, Shi Z, Li C, Guo F, Li S, Li Y, Na L, Sun C. Ursolic acid increases energy expenditure through enhancing free fatty acid uptake and β-oxidation via an UCP3/AMPKdependent pathway in skeletal muscle. Mol Nutr Food Res. 2015; 59:1491-1503.
Jose G. C. Cione et. al; A double-blind, randomized, placebo-controlled trial Clinical Nutrition ESPEN Volume 44, August 2021, Pages 143-149.
Kadasah, S. F.; Radwan, M. O. Overview of Ursolic Acid Potential for the Treatment of Metabolic Disorders, Autoimmune Diseases, and Cancers via Nuclear Receptor Pathways. Biomedicines 2023, 11, 2845.
Kazmi I, Afzal M, Rahman S, Iqbal M, Imam F, Anwar F. Antiobesity potential of ursolic acid stearoyl glucoside by inhibiting pancreatic lipase. Eur J Pharmacol. 2013; 709:28-36.
Kunkel S D, Elmore C J, Bongers K S, Ebert S M, Fox D K, Dyle M C, Bullard S A, Adams C M. Ursolic acid increases skeletal muscle and brown fat and decreases diet-induced obesity, glucose intolerance and fatty liver disease. PLoS One. 2012; 7:e39332.
Li S, Liao X, Meng F, Wang Y, Sun Z, Guo F, Li X, Meng M, L Y, Sun C. Therapeutic role of ursolic acid on ameliorating hepatic steatosis and improving metabolic disorders in highfat diet-induced nonalcoholic fatty liver disease rats. PLoS One. 2014; 9:e86724.
Luo, Q.; Wei, Y.; Lv, X.; Chen, W.; Yang, D.; Tuo, Q. The Effect and Mechanism of Oleanolic Acid in the Treatment of Metabolic Syndrome and Related Cardiovascular Diseases. Molecules 2024, 29, 758.
Oluwatosin Omodara et. al; Anti-inflammatory Effect of Ursolic Acid on Male Wistar Rats with High-carbohydrate High-fat Diet-induced Metabolic Syndrome. J Med B Sci Res Vol. 4 No. 3-4 (2023).
Omodara et. al; Ursolic acid prevents the development of metabolic syndrome in male Wistar rats fed a high-carbohydrate high-fat diet. 10.0 J. Afr. Ass. Physiol. Sci. 10 (1): 1-12, 2022.
Ramirez-Rodriguez A M, Gonzalez-Ortiz M, Martinez-Abundis E, Acuna Ortega N. Effect of Ursolic Acid on Metabolic Syndrome, Insulin Sensitivity, and Inflammation. J Med Food. 2017 September; 20(9):882-886.
Wu D M, Lu J, Zhang Y Q, Zheng Y L, Hu B, Cheng W, Zhang Z F, Li M Q. Ursolic acid improves domoic acid-induced cognitive deficits in mice. Toxicol Appl Pharmacol. 2013; 271:127-136.
Yu S G, Zhang C J, Xu X E, Sun J H, Zhang L, Yu P F. Ursolic acid derivative ameliorates streptozotocin-induced diabetic bone deleterious effects in mice. Int J Clin Exp Pathol. 2015; 8:3681-3690.
Zhang Y, Song C, Li H, Hou J, Li D. Ursolic acid prevents augmented peripheral inflammation and inflammatory hyperalgesia in high-fat diet-induced obese rats by restoring downregulated spinal PPARα. Mol Med Rep. 2016; 13:5309-5316.

I claim:

1. A method for preparing a composition for the treatment of a metabolic disorder, wherein the method comprises:
(i) blending (a) one or more of 3β-hydroxyurs-12-en-28-oic acid (UA), 3β-hydroxyolean-12-en-28-oic acid (OA), and salts thereof with (b) at least one modulator extract selected from the group consisting of piperine, capsaicin, and gingerol, and (c) a water-soluble polymer having a glass transition temperature lower than 170° C.;
(ii) melt extruding the blend at a temperature about 60° C. to about 180° C. to obtain an extrusion product;
(iii) grinding and mixing the extrusion product with a carrier or excipient to produce the composition.

2. The method of claim 1, wherein the composition is free from any solvent.

3. The method of claim 1, wherein UA and/or OA is in amorphous form in the composition.

4. The method of claim 1, wherein the UA and/or OA is a component of a botanical extract.

5. The method of claim 1, wherein the at least one modulator extract is piperine of formula:

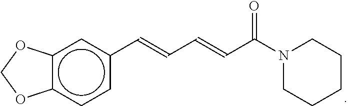

6. The method of claim 1, wherein the water-soluble polymer is selected from the group consisting of polyvinyl caprolactam, polyvinyl acetate-polyethylene glycol graft copolymer, polyvinylpyrrolidone-vinyl acetate copolymer, polyethylene glycol, amino methacrylate copolymer, hypromellose acetate succinate and combinations thereof.

7. The method of claim 1, wherein the water-soluble polymer is poly vinylpyrrolidonevinyl acetate copolymer.

8. The method of claim 1, wherein UA and/or OA is about 1% to 50% of the weight of the composition.

9. The method of claim 1, wherein the at least one modulator extract is about 0.1 to about 25% of the weight of the composition.

10. The method of claim 1, wherein the water-soluble polymer is about 5% to about 90% of the weight of the composition.

11. The method of claim 1, wherein the blend of (i) is melted while it passes through at least four heating zones of an extruder, wherein a temperature of each heating zone is sequentially lowered.

12. A pharmaceutical composition comprising:
(a) at least one of the following:
UA, an isomer, or a salt thereof; and
OA, an isomer or a salt thereof;
(b) at least one modulator extract selected from the group consisting of piperine, capsaicin, and gingerol;
(c) a water-soluble polymer having a glass transition temperature lower than 170° C.; and
(d) a pharmaceutically acceptable carrier.

13. The pharmaceutical composition of claim 12, wherein the UA is a compound of formula:

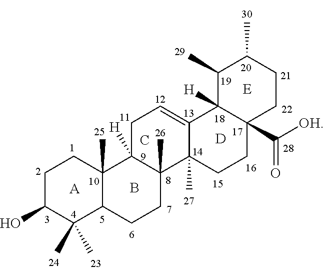

14. The pharmaceutical composition of claim 12, wherein the OA is a compound of formula:

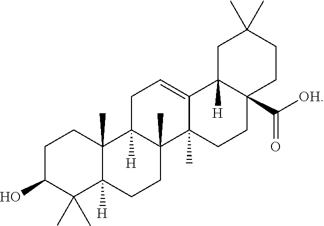

15. The pharmaceutical composition of claim 12, wherein the at least one modulator extract is piperine of formula:

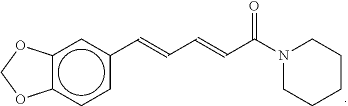

16. The pharmaceutical composition of claim 12, wherein the composition comprises a water-soluble polymer selected from the group consisting of polyvinyl caprolactam, polyvinyl acetate-polyethylene glycol graft copolymer, polyvinylpyrrolidone-vinyl acetate copolymer, polyethylene glycol, amino methacrylate copolymer, and hypromellose acetate succinate.

17. The pharmaceutical composition of claim 12, wherein the water-soluble polymer is poly vinylpyrrolidonevinyl acetate copolymer.

18. The pharmaceutical composition of claim 12, wherein UA and/or OA is about 1% to 50% of the weight of the composition.

19. The pharmaceutical composition of claim 12, wherein the at least one modulator extract is about 0.1 to about 25% of the weight of the composition.

20. The pharmaceutical composition of claim 12, wherein the water-soluble polymer is about 5% to about 90% of the weight of the composition.

21. The pharmaceutical composition of claim 12, which is in a form of a capsule, tablet, chewable tablet, soft chew, gummy, lozenge, bar, oral paste, suppository, powder, or an adjunct to food or feed.

22. A method for treating a metabolic disorder in a subject, the method comprising administering to the subject a therapeutically effective amount of the pharmaceutical composition of claim 12.

23. The method of claim 22, wherein the subject is human.

24. The method of claim 22, wherein the metabolic disorder is metabolic syndrome.

25. A supplement comprising the pharmaceutical composition of claim 12.
